(19) United States
(12) Patent Application Publication
(10) Pub. No.: US 2013/0167617 A1
(43) Pub. Date: Jul. 4, 2013

(54) METHOD FOR EVALUATING THE QUANTITY OF METHANE PRODUCED BY A RUMINANT USED FOR MEAT PRODUCTION

(71) Applicants: Pierre Weill, Vern Sur Seiche (FR); Guillaume Chesneau, Luitre (FR); Aude Guerin, Saint Hilaire Des Landes (FR)

(72) Inventors: Pierre Weill, Vern Sur Seiche (FR); Guillaume Chesneau, Luitre (FR); Aude Guerin, Saint Hilaire Des Landes (FR)

(73) Assignee: VALOREX, Combourtille (FR)

(21) Appl. No.: 13/807,573

(22) PCT Filed: Jun. 23, 2011
(86) PCT No.: PCT/EP2011/060556

§ 371 Date: Mar. 14, 2013

(30) Foreign Application Priority Data
Jun. 29, 2010 (FR) 1055243

(51) Int. Cl. G01N 33/12

(52) U.S. Cl. CPC G01N 33/12
USPC 73/31.05

(57) ABSTRACT

The present invention relates to a method for determining the quantity of methane produced by a meat ruminant, such as a bovine, i.e., an animal raised and then slaughtered for the sale of its meat, characterized in that it consists in determining the quantity of at least one fatty acid (FA) contained in a reference tissue, namely muscle or adipose tissue, sampled from said ruminant after its death (in grams of fatty acids per kilogram of tissue) and calculating said quantity of methane (in grams of CH₄ per kilogram of meat from the animal) according to an equation that is a function of said quantity of said FA and the category, age and weight of said animal, wherein the latter three criteria are determined at the time of the slaughter of said animal.

The present invention relates to a method for determining the quantity of methane generated from the rearing of ruminants raised for their meat using the fatty acid composition of meat lipids.

Methane is a greenhouse gas which is involved in global warming.

Its global warming potential is 21 times that of carbon dioxide. Thus, according to experts, methane contributes 20% of all greenhouse gases leading to global warming.

Half of this methane comes from agriculture (10% of greenhouse gases globally).

Additionally, most methane of agricultural origin (70% in Europe) is enteric methane emitted by livestock during their digestion process.

In France, ruminants are the source of 98% of this enteric methane.

Indeed, fermentation processes in ruminants produce high methane emissions.

Approximately half of this enteric methane from ruminants comes from dairy cows and the other half from herds reared specifically for meat production.

Thus, many experts call for a decrease in the consumption of meat from ruminants (mainly beef) whereas other experts call for modes of production that emit less methane.

To validate modes of production that emit less methane, it is necessary to have methane measurements linked to the mode of production.

According to the type of production, the quantity of methane emitted per kilo of meat produced can vary from 1 to 6.

The existing methods for determining this quantity of methane produced per kilo of meat are difficult to implement.

They are indeed based on measurements at experimental farms (calorimetric chamber methods, indirect measurements using sulfur hexafluoride) which prove to be impossible to set up systematically.

Other methods use prediction equations which comprise, to be precise, a host of individual data specific to meat-producing animals, such as their age, their growth rate, their weight, the quantity of rations ingested during their life and the composition of these rations at each stage of their life.

In all cases, to date, it is impossible to know the quantity of methane emitted during the production of a kilogram of meat without precise data on the animal from which this meat is produced.

The present Applicant has filed a French patent (08 54230) relating to a method for determining the methane produced per liter of milk from dairy cows which makes use of a rapid method linking the fatty acid composition of milk to methane production.

In this context, the problem is relatively simple for milk cows since there is a direct link between the quantity of methane emitted daily by the cow and the quantity of fatty acids present daily in the milk.

Moreover, samples of milk are taken from living animals whose performances are known.

Today, there is thus still an unmet need for a method for determining the so-called “methane footprint” of meat and, consequently, for directing producers toward methods respectful of constraints related to global warming.

The present invention aims at responding to this need.

Thus, the invention relates to a method for determining the quantity of methane produced by a meat ruminant, such as a bovine, i.e., an animal raised and then slaughtered for the sale of its meat, characterized in that it consists in measuring the quantity of at least one fatty acid (FA) contained in a reference tissue, namely muscle or adipose tissue, sampled from said ruminant after its death (in grams of fatty acids per kilogram of tissue) and calculating said quantity of methane (in grams of CH₄ per kilogram of meat from the animal) according to an equation that is a function of said quantity of said FA and the category, age and weight of said animal, wherein the latter three criteria are determined at the time of the slaughter of said animal.

Thus, by virtue of this method, the “methane footprint” of meat can be measured rapidly, inexpensively and systematically through the knowledge of just a few elements, namely:
the age of the animal from which the tissue comes and its category (steer, heifer, cow, etc.),
a tissue sample from said animal (sample of the longissimus dorsi muscle, for example).

The animal's age always appears among the traceability data which accompany meat carcasses.

The tissue sample is used to measure the content of at least one fatty acid in the lipids of said sample.

It should be noted that many referenced works link ruminal methanogenesis mechanisms to those of lipogenesis in liver and adipose tissue.

Moreover, experimental data are available which link the mode of production of animals raised for their meat (type of rearing and composition of rations) to the lipid composition of meats, in particular data relative to:
i. Percentage of lipids in the sample by ether extraction;
ii. Fatty acid profile of said lipids measured by gas chromatography or other methods.

Lastly, a database is used to predict the content of minor fatty acids from certain major fatty acids and groups of fatty acids in samples of ruminant meat or adipose tissue.

According to other advantageous and nonrestrictive characteristics of this method:
said quantity is calculated according to the equation:

CH₄(g/kg meat of the animal)=[[[(Age in months)*Coeff 1]+Coeff 2]*1000*Lipid content (in %)*FA content (in % of total lipids)]*1000/Weight of the animal (in kg of meat),
wherein:
the coefficients Coeff 1 and Coeff 2 are numbers whose value is a function of the nature of the reference tissue and the category of the animal;
said reference tissue is the longissimus dorsi muscle;
said measuring of FA quantity is carried out by direct analysis of the reference tissue;
said measuring of FA quantity is carried out by analysis of another tissue, and then deduction of FA quantity of said reference muscle by a prediction equation;
said FA is palmitic acid (C16:0);
said other tissue is selected from flank, skirt and silverside and the quantity of palmitic acid in the longissimus dorsi (C16:0_{LD}) is given by one of the following equations:

C16:0_{LD}=0.884*C16:0_{Flank}+2.240(r²=0.855,n=48,p<0.001);

C16:0_{LD}=1.053*C16:0_{Skirt}+1.076(r²=0.78,n=67,p<0.001);

C16:0_{LD}=0.948*C16:0_{Silverside}+2.095(r²=0.70,n=25,p<0.001);

equations wherein:
C16:0_{Flank}, C16:0_{Skirt} and C16:0_{Silverside} are the palmitic acid contents of the corresponding tissues, r is the correlation coefficient, n is the number of samples tested and p is the significance level;
said FA is different from palmitic acid, but strongly correlated with palmitic acid, with a significance level (p) less than 0.01;
said Coefficients 1 and 2 have the following values:

| | Coeff 1 | Coeff 2 |
|---|---|---|
| Young bovine | 1.511 ± 0.506 | −13.782 ± 5.0615 |
| Heifer | 0.555 ± 0.1905 | −4.807 ± 1.907 |
| Steer | 0.885 ± 0.278 | −7.522 ± 2.779 |
| Nursing cow | 0.582 ± 0.235 | 0 |

said coefficients have the following values:

| | Coeff 1 | Coeff 2 |
|---|---|---|
| Young bovine | 1.507 | −13.792 |
| Heifer | 0.556 | −5.108 |
| Steer | 0.8848 | −7.552 |
| Nursing cow | 0.582 | 0.000 |

Throughout the present application, the following terms are defined as below:
heifer: female not having had a calf;
nursing cow: female having had at least one calf and whose milk was used to feed it;
young bovine: uncastrated male younger than 2 years;
steer: castrated male older than 2 years.

Other characteristics and advantages of the invention will appear upon consideration of the following detailed description of certain embodiments.

The particular problems of ruminants, in particular bovines, reared for their meat are as follows.

During rumination processes, carbohydrates present in ruminant feed are fermented by microbe populations present in the rumen.

Plant polysaccharides from feed are broken down into monosaccharides which are then fermented with production of organic acids (volatile fatty acids, or VFAs), hydrogen and carbon dioxide.

Two principal pathways coexist in the rumen:
i. The pathway leading to the formation of acetic acid (abbreviated C2 for two carbon atoms) and butyric acid (abbreviated C4 for four carbon atoms).

This fermentation pathway produces hydrogen.

The hydrogen thus produced is then evacuated mainly in the form of methane (CH₄) during ruminant eructation.
ii. The pathway leading to the formation of propionic acid (abbreviated C3 for three carbon atoms).

This pathway, in contrast, consumes hydrogen present in the rumen.

Thus, methane production is a physiological phenomenon related to microbial fermentation processes in the rumen of polygastric animals.

It has long been known that the equilibrium of these two pathways is highly variable as a function of animal feed. Thus, the composition of animal feed promotes different fermentation pathways:
consumers of hydrogen, thus reducing methane production in the C3 pathway;

or
producers of hydrogen, thus responsible for methane production in the C2 and C4 pathways.

Many characteristics of feed must be taken into account to predict an orientation toward the C2 and C4 pathways or the C3 pathway. Examples of such characteristics include the fiber content of feed, the quantity of concentrates, cellulose content, starch content, lipid content, etc.

Among the lipids present in ruminant feed, a polyunsaturated fatty acid of the n−3 or omega-3 family, alpha-linolenic acid (nomenclature C18:3 n−3), occupies a special place for several reasons:
It is the principal fatty acid of grazed forage (up to 70% of fatty acids in spring grass).
It has three unsaturations which are for the most part hydrogenated in the rumen, thus consuming a small fraction of the hydrogen produced by the C2 and C4 pathway and producing intermediate compounds of biohydrogenation and stearic acid (C18:0).
Its inclusion in feed orients fermentation more toward C3 and less toward C2 and C4.
For more than 30 years, an abundance of referenced works have indicated that its inclusion in feed reduces the quantity of methane produced by ruminants under experimental conditions (calorimetric chamber).
The mechanism of this reduction in methane involves a toxic effect of this fatty acid (and/or some of its derivatives from biohydrogenation in the rumen) on certain rumen microbes involved in producing hydrogen and thus in the first steps of methanogenesis.
Lastly, omega-3 alpha-linolenic acid is a so-called “essential” fatty acid for animals because its synthesis uses enzymes (delta-12 and delta-15 desaturases) present only in the plant kingdom. If this FA is found in meat, it inevitably comes from feed.

Thus, if this fatty acid (or a derivative thereof) is found in meat, it can be regarded as a marker for feed practices that discourage methane production, because these feed practices promote the hydrogen-consuming C3 pathway at the expense of the hydrogen-producing C2 and C4 pathways and thus the CH4 pathway.

Lipogenesis is the synthesis of lipids (and particularly fatty acids) from precursors which are essentially carbohydrates in monogastrics and essentially the volatile fatty acid (VFA) acetic acid (C2) in ruminants.

Thus, the element essential to lipid synthesis in ruminants raised for meat is acetic acid (C2), whose ruminal production is accompanied by emission of CH₄.

The fatty acids present in ruminant tissues can have two origins, namely:
Endogenous, when they result from lipogenesis from the acetic acid (C2) precursor. These are saturated or monounsaturated fatty acids.
Exogenous, when they come from feed and are incorporated in triglycerides or other lipid fractions (phospholipids). These notably include exclusively exogenous polyunsaturated fatty acids (since no animals have the enzymes necessary to synthesize them).

Thus, C18:3 n−3 linolenic acid and acetic acid (C2 VFA) are at the junction of mechanisms of methanogenesis and lipogenesis.

Alpha-linolenic acid, because its presence (or that of its omega-3 derivatives) in ruminant lipids is an indication of its presence in feed and thus of a reduction in the production of acetic acid and methane.

Acetic acid, because its presence is necessary for the synthesis of most saturated fatty acids and because its production is always accompanied by methane production.

Palmitic acid predominantly results from endogenous synthesis from the C2 precursor. The quantity of endogenous palmitic acid is directly related to the availability of acetic acid precursor and thus to the ruminal production of hydrogen and then of methane.

Reduction in the palmitic acid content of meat is thus always related to a reduction in methane emissions if alpha-linolenic acid (principal fatty acid in ruminant feed) is the predominant fatty acid in the feed.

The lipids of ruminant meat are either:
structural lipids, constitutive of cell membranes of muscle and other organs; or
reserve lipids, present in triglycerides in adipose tissue internal (marbling in meat) or external to muscles (or other organs).

Adipose tissue is an “energy reserve” for the animal; adipose tissue fatty acids (FAs) are thus regularly mobilized as the animal ages. Other FAs from feed (endogenous via C2) or from exogenous sources are in turn incorporated in adipose tissue.

In all cases, the quantity of lipids present in a given muscle reflects the intensity of endogenous lipogenesis mechanisms (thus production of C2 and consequently of CH₄) but also the age and history of the animal, i.e., the intensity of its adipose tissue regeneration mechanisms.

The quantity and nature of the fatty acids present in the meat of an animal of a known age are themselves a function of the nature of the animal's feed. It thus reflects the animal's methane production during its life.

### Examples of Implementation of the Method

When the animal is slaughtered, a meat sample is taken in a standardized way, such as a sample of muscle at the sixth rib, for example.

The sample is then analyzed to determine:
its lipid content;
the fatty acid profile of its total lipids.

This analysis is carried out by ether extraction for total lipids and by gas chromatography for fatty acids.

Furthermore, standard traceability data will give the animal's age, sex and breed.

Thus, the following data is available (examples):

Animal 1: Longissimus Dorsi Muscle

Animal type: young bovine of the Limousin breed (beef breed).

Age: 17 months.

Weight: 400 kg of meat (=carcass weight in kg*meat yield in %).

Lipid content of the sample: 2.5% (by weight).

C16:0 content: 25.0% (of total FAs).

Then, the quantity of methane emitted is calculated as follows:

CH₄(g/kg of meat)=[[[(Age in months)*Coeff 1]+Coeff 2]*1000*Lipid content (in %)*C16:0 content (in % of total FAs)]*1000/Weight (in kg of meat).

For young bovines of beef breeds, the coefficients Coeff 1 and Coeff 2 have the following values: Coeff 1=1.511 and Coeff 2=−13.782.

Result: CH₄ (g/kg of meat)=185 g.

Animal 1: Skirt muscle

Animal type: young bovine of the Limousin breed (beef breed).

Age: 17 months.

Weight: 400 kg of meat (=carcass weight in kg*meat yield in %).

Lipid content of the sample: 7.5% (by weight).

C16:0 content: 24.5% (of total FAs).

Quantity of methane emitted:

CH₄(g/kg of meat)=[[[(Age in months)*Coeff 1]+Coeff 2]*1000*Lipid content (in %)*C16:0 content (in % of total FAs)]*1000/Weight (in kg of meat).

For young bovines of beef breeds: Coeff 1=0.514 and Coeff 2=−4.70.

Result: CH₄ (g/kg of meat)=185 g.

Animal 2: Longissimus dorsi muscle

Animal type: young bovine of the Limousin breed (beef breed).

Age: 17 months.

Weight: 400 kg of meat (=carcass weight in kg*meat yield in %).

Lipid content of the sample: 2.5% (by weight).

C16:0 content: 23.0% (of total FAs).

Quantity of methane emitted:

CH₄(g/kg of meat)=[[[(Age in months)*Coeff 1]+Coeff 2]*1000*Lipid content (in %)*C16:0 content (in % of total FAs)]*1000/Weight (in kg of meat).

For young bovines of beef breeds: Coeff 1=1.511 and Coeff 2=−13.782.

Result: CH₄ (g/kg of meat)=170 g.

Animal 2: Skirt muscle

Animal type: young bovine of the Limousin breed (beef breed).

Age: 17 months.

Weight: 400 kg of meat (=carcass weight in kg*meat yield in %).

Lipid content of the sample: 7.5% (by weight).

C16:0 content: 22.5% (of total FAs).

Quantity of methane emitted:

CH₄(g/kg of meat)=[[[(Age in months)*Coeff 1]+Coeff 2]*1000*Lipid content (in %)*C16:0 content (in % of total FAs)]*1000/Weight (in kg of meat).

For young bovines of beef breeds: Coeff 1=0.514 and Coeff 2=−4.70.

Result: CH₄ (g/kg of meat)=170 g.

From the tests and from data of the referenced works, said coefficients were calculated for each category of animal and each muscle.

Preferentially, the coefficients for the longissimus dorsi muscle have the following values:

| | Coeff 1 | Coeff 2 |
|---|---|---|
| Young bovine | 1.511 | −13.782 |
| Heifer | 0.555 | −4.807 |
| Steer | 0.885 | −7.522 |
| Nursing cow | 0.582 | 0.000 |

For skirt muscle, the coefficients preferentially have the following values:

| | Coeff 1 | Coeff 2 |
|---|---|---|
| Young bovine | 0.5142 | −4.700 |
| Heifer | 0.3356 | −2.9042 |
| Steer | 0.3011 | −2.5596 |
| Nursing cow | 0.2671 | 0.000 |

As an example, the prediction equations used when the muscle tested is not the longissimus dorsi and the fatty acid is palmitic acid are given below. Said other tissue can be selected from flank, skirt and silverside and the quantity of palmitic acid in longissimus dorsi

C16:0_{LD}=0.884*C16:0_{Flank}+2.240(r²=0.855,n=48,p<0.001);

C16:0_{LD}=1.053*C16:0_{Skirt}+1.076(r²=0.78,n=67,p<0.001);

C16:0_{LD}=0.948*C16:0_{Silverside}+2.095(r²=0.70,n=25,p<0.001);

equations wherein C16:0_{Flank}, C16:0_{Skirt} and C16:0_{Silverside} are the palmitic acid contents of the corresponding tissues, r is the correlation coefficient, n is the number of samples tested and p is the significance level.

Moreover, table 1 below presents a correlation (prediction) matrix for calculating, by a Y=aX+b equation with Y=C16:0 (muscle i) and X=quantity of another fatty acid (same muscle i), the quantity of palmitic acid in said muscle. Here, the muscle i is longissimus dorsi.

TABLE 1
Muscle = Raw longissimus dorsi (mg/100 g)

| | Y | | | | | | | |
|---|---|---|---|---|---|---|---|---|
| X | Mean | SD | r | r² | p | n | a | b |
| C16:0 | 789.37 | 479.66 | 0.00 | 0.00 | 0.00 | 0 | 0.000 | 0.000 |
| SFA | 1554.95 | 837.47 | 0.98 | 0.97 | 0.00 | 208 | 0.563 | −86.562 |
| MUFA | 1334.78 | 807.28 | 0.98 | 0.96 | 0.00 | 208 | 0.584 | 10.441 |
| C18:1 | 1201.25 | 715.86 | 0.98 | 0.96 | 0.00 | 208 | 0.656 | 1.199 |
| PUFA | 189.73 | 63.53 | 0.54 | 0.29 | 0.00 | 208 | 4.050 | 21.054 |
| PUFA n-3 | 34.10 | 18.36 | 0.47 | 0.22 | 0.00 | 208 | 12.309 | 369.697 |
| PUFA n-3-LC | 13.88 | 8.89 | 0.34 | 0.11 | 0.00 | 208 | 18.152 | 537.509 |
| CLA | 9.40 | 7.85 | 0.72 | 0.52 | 0.00 | 208 | 44.247 | 373.670 |
| ALA | 20.22 | 11.44 | 0.49 | 0.24 | 0.00 | 208 | 20.754 | 369.742 |
| C14:0 | 77.52 | 51.75 | 0.98 | 0.96 | 0.00 | 208 | 9.058 | 87.178 |
| C15:0 iso | 5.69 | 3.53 | 0.87 | 0.76 | 0.00 | 208 | 118.806 | 113.845 |
| C15:0 | 13.18 | 6.88 | 0.91 | 0.82 | 0.00 | 208 | 63.306 | −45.021 |
| C16:0 iso | 7.29 | 3.94 | 0.76 | 0.57 | 0.00 | 208 | 92.233 | 117.169 |
| C16:1 | 90.04 | 68.57 | 0.95 | 0.90 | 0.00 | 208 | 6.637 | 191.789 |
| C17:0 iso | 12.53 | 6.50 | 0.92 | 0.84 | 0.00 | 208 | 67.753 | −59.374 |
| C16:0 | 789.37 | 479.66 | 0.00 | 0.00 | 0.00 | 0 | 0.000 | 0.000 |
| C17:0 | 31.35 | 16.62 | 0.93 | 0.86 | 0.00 | 208 | 26.840 | −51.935 |
| C17:1 | 18.66 | 11.95 | 0.96 | 0.91 | 0.00 | 208 | 38.366 | 73.322 |
| C18:0 iso | 5.08 | 2.88 | 0.76 | 0.58 | 0.00 | 208 | 126.523 | 147.296 |
| C18:0 | 565.84 | 273.92 | 0.88 | 0.77 | 0.00 | 208 | 1.539 | −81.489 |
| C18:2 | 29.13 | 17.45 | 0.66 | 0.44 | 0.00 | 208 | 18.273 | 257.130 |
| C18:3 | 3.32 | 1.80 | 0.92 | 0.84 | 0.00 | 137 | 255.259 | 45.215 |
| C17:0 anteiso | 20.22 | 9.99 | 0.86 | 0.73 | 0.00 | 207 | 41.213 | −43.617 |
| C15:0 anteiso | 7.43 | 3.81 | 0.79 | 0.62 | 0.00 | 207 | 99.460 | 52.245 |
| C14:1 | 11.56 | 11.03 | 0.87 | 0.76 | 0.00 | 207 | 37.726 | 356.361 |
| C14:0 iso | 2.96 | 1.71 | 0.92 | 0.85 | 0.00 | 102 | 245.730 | 38.147 |
| C12:0 | 3.02 | 1.68 | 0.97 | 0.94 | 0.00 | 190 | 276.593 | −58.359 |
| C10:0 | 2.64 | 1.69 | 0.83 | 0.69 | 0.00 | 107 | 218.752 | 155.799 |

Furthermore, in the following tables 2 to 4 are given the correlation matrices for calculating the quantity of palmitic acid of said muscle i, by coupling a Y=aX+b equation, with Y=C16:0 (muscle j) and X=quantity of another fatty acid (same muscle i), and a prediction equation for C16:0 of the muscle i from C16:0 of the muscle j.

Here, the muscle i is longissimus dorsi and the muscle j is flank, skirt and silverside, respectively.

TABLE 2
Muscle = Flank (mg/100 g)

| | Y | | | | | | | |
|---|---|---|---|---|---|---|---|---|
| X | Mean | SD | r | r² | p | n | a | b |
| C16:0 | 603.75 | 350.03 | 0.00 | 0.00 | 0.00 | 0 | 0.000 | 0.000 |
| SFA | 1179.37 | 631.01 | 0.99 | 0.99 | 0.00 | 48 | 0.551 | −46.069 |
| MUFA | 1052.97 | 657.66 | 0.96 | 0.92 | 0.00 | 48 | 0.511 | 65.408 |
| C18:1 | 933.58 | 580.92 | 0.96 | 0.91 | 0.00 | 48 | 0.576 | 65.854 |
| PUFA | 184.95 | 81.65 | 0.67 | 0.45 | 0.00 | 48 | 2.887 | 69.750 |
| PUFA n-3 | 25.75 | 10.26 | 0.59 | 0.35 | 0.00 | 48 | 20.178 | 84.225 |
| PUFA n-6 | 127.50 | 59.68 | 0.57 | 0.33 | 0.00 | 48 | 3.359 | 175.542 |
| PUFA n-3-LC | 11.02 | 3.82 | 0.50 | 0.25 | 0.00 | 48 | 45.809 | 98.863 |
| CLA | 5.62 | 4.02 | 0.86 | 0.74 | 0.00 | 48 | 75.009 | 181.942 |
| ALA | 14.73 | 7.66 | 0.54 | 0.30 | 0.00 | 48 | 24.854 | 237.775 |
| LA | 108.15 | 51.69 | 0.59 | 0.34 | 0.00 | 48 | 3.972 | 174.199 |
| C14:0 | 67.67 | 44.32 | 0.98 | 0.97 | 0.00 | 48 | 7.776 | 77.544 |
| C15:0 iso | 3.60 | 2.12 | 0.80 | 0.64 | 0.00 | 48 | 132.329 | 126.805 |
| C15:0 | 12.51 | 6.63 | 0.92 | 0.85 | 0.00 | 48 | 48.655 | −4.782 |
| C16:0 iso | 6.85 | 3.57 | 0.85 | 0.73 | 0.00 | 48 | 83.802 | 29.347 |
| C16:1 | 76.11 | 50.31 | 0.98 | 0.96 | 0.00 | 48 | 6.802 | 86.044 |
| C17:0 iso | 10.88 | 5.60 | 0.85 | 0.73 | 0.00 | 48 | 53.239 | 24.653 |
| C17:0 | 27.53 | 16.61 | 0.86 | 0.73 | 0.00 | 48 | 18.054 | 106.660 |
| C17:1 | 17.45 | 12.07 | 0.87 | 0.76 | 0.00 | 48 | 25.304 | 162.317 |
| C18:0 iso | 5.16 | 3.13 | 0.87 | 0.76 | 0.00 | 48 | 97.778 | 99.249 |
| C18:0 | 394.37 | 190.99 | 0.94 | 0.89 | 0.00 | 48 | 1.729 | −78.148 |
| C18:2 | 24.16 | 15.70 | 0.66 | 0.44 | 0.00 | 48 | 14.809 | 246.016 |
| C18:2 cj | 5.62 | 4.02 | 0.86 | 0.74 | 0.00 | 48 | 75.009 | 181.942 |
| C18:3 | 2.00 | 1.50 | 0.61 | 0.37 | 0.00 | 46 | 142.480 | 314.902 |
| C17:0 anteiso | 22.08 | 14.68 | 0.80 | 0.64 | 0.00 | 48 | 19.121 | 181.574 |
| C15:0 anteiso | 6.86 | 3.77 | 0.83 | 0.69 | 0.00 | 48 | 76.782 | 76.753 |
| C14:1 | 11.93 | 10.06 | 0.93 | 0.87 | 0.00 | 48 | 32.377 | 217.450 |
| C14:0 iso | 2.02 | 1.20 | 0.86 | 0.73 | 0.00 | 22 | 217.297 | 155.501 |
| C12:0 | 2.41 | 1.33 | 0.98 | 0.96 | 0.00 | 48 | 257.569 | −17.080 |
| C10:0 | 2.22 | 1.45 | 0.92 | 0.84 | 0.00 | 44 | 222.915 | 107.146 |

TABLE 3
Muscle = Skirt (mg/100 g)

| | Y | | | | | | | |
|---|---|---|---|---|---|---|---|---|
| X | Mean | SD | r | r² | p | n | a | b |
| C16:0 | 1791.78 | 807.61 | 0.00 | 0.00 | 0.00 | 0 | 0.000 | 0.000 |
| SFA | 4310.14 | 1731.80 | 0.98 | 0.97 | 0.00 | 70 | 0.459 | −184.254 |
| MUFA | 3092.75 | 1456.98 | 0.94 | 0.89 | 0.00 | 70 | 0.522 | 177.956 |
| C18:1 | 2845.68 | 1341.17 | 0.94 | 0.88 | 0.00 | 70 | 0.566 | 180.761 |
| PUFA | 494.93 | 152.88 | 0.51 | 0.26 | 0.00 | 70 | 2.708 | 451.492 |
| PUFA n-3 | 69.11 | 27.72 | 0.46 | 0.21 | 0.00 | 70 | 13.305 | 872.315 |
| CLA | 20.61 | 15.06 | 0.77 | 0.60 | 0.00 | 70 | 41.411 | 938.128 |
| ALA | 49.37 | 23.52 | 0.48 | 0.23 | 0.00 | 70 | 16.486 | 977.860 |
| C14:0 | 190.48 | 88.86 | 0.97 | 0.93 | 0.00 | 70 | 8.781 | 119.263 |
| C15:0 iso | 15.27 | 7.57 | 0.85 | 0.72 | 0.00 | 70 | 90.646 | 407.941 |
| C15:0 | 37.96 | 14.72 | 0.83 | 0.69 | 0.00 | 70 | 45.544 | 63.056 |
| C16:0 iso | 24.06 | 10.65 | 0.82 | 0.68 | 0.00 | 70 | 62.359 | 291.505 |
| C16:1 | 150.14 | 72.08 | 0.94 | 0.89 | 0.00 | 70 | 10.583 | 202.871 |
| C17:0 iso | 35.26 | 13.80 | 0.88 | 0.77 | 0.00 | 70 | 51.524 | −25.185 |
| C17:0 | 102.57 | 42.63 | 0.90 | 0.82 | 0.00 | 70 | 17.121 | 35.782 |
| C17:1 | 43.78 | 20.96 | 0.88 | 0.78 | 0.00 | 70 | 33.949 | 305.582 |
| C18:0 iso | 16.64 | 7.45 | 0.90 | 0.81 | 0.00 | 70 | 97.735 | 165.180 |
| C18:0 | 1944.47 | 738.37 | 0.93 | 0.86 | 0.00 | 70 | 1.013 | −176.922 |
| C18:2 | 74.78 | 45.87 | 0.54 | 0.30 | 0.00 | 70 | 9.579 | 1075.396 |
| C18:2 cj | 20.61 | 15.06 | 0.77 | 0.60 | 0.00 | 70 | 41.411 | 938.128 |
| C18:3 | 7.40 | 3.66 | 0.88 | 0.77 | 0.00 | 69 | 192.310 | 353.173 |
| C17:0 anteiso | 67.74 | 26.74 | 0.76 | 0.58 | 0.00 | 70 | 22.964 | 236.193 |
| C15:0 anteiso | 24.91 | 8.96 | 0.71 | 0.50 | 0.00 | 70 | 63.787 | 202.580 |
| C14:1 | 17.75 | 11.60 | 0.83 | 0.69 | 0.00 | 70 | 57.833 | 765.313 |
| C14:0 iso | 7.91 | 3.56 | 0.78 | 0.61 | 0.00 | 49 | 176.833 | 508.393 |
| C12:0 | 7.58 | 3.13 | 0.98 | 0.96 | 0.00 | 64 | 249.034 | −167.120 |
| C10:0 | 7.03 | 3.87 | 0.90 | 0.80 | 0.00 | 57 | 186.333 | 420.298 |

TABLE 4
Muscle = Silverside (mg/100 g)

| | Y | | | | | | | |
|---|---|---|---|---|---|---|---|---|
| X | Mean | SD | r | r² | p | n | a | b |
| C16:0 | 464.52 | 227.74 | 0.00 | 0.00 | 0.00 | 0 | 0.000 | 0.000 |
| SFA | 822.70 | 391.97 | 0.99 | 0.99 | 0.00 | 25 | 0.578 | −11.061 |
| MUFA | 944.87 | 448.35 | 0.98 | 0.96 | 0.00 | 25 | 0.498 | −6.176 |
| C18:1 | 827.98 | 392.43 | 0.98 | 0.96 | 0.00 | 25 | 0.569 | −6.427 |
| PUFA | 84.94 | 29.49 | 0.67 | 0.45 | 0.00 | 25 | 5.157 | 26.474 |
| CLA | 6.57 | 4.09 | 0.74 | 0.54 | 0.00 | 25 | 41.014 | 195.174 |
| ALA | 5.42 | 2.42 | 0.50 | 0.25 | 0.01 | 25 | 47.380 | 207.744 |
| LA | 35.74 | 10.74 | 0.70 | 0.49 | 0.00 | 25 | 14.872 | −66.985 |
| C14:0 | 40.88 | 22.87 | 0.96 | 0.93 | 0.00 | 25 | 9.608 | 71.761 |
| C15:0 iso | 3.32 | 1.94 | 0.92 | 0.85 | 0.00 | 25 | 108.175 | 105.723 |
| C15:0 | 7.03 | 3.70 | 0.95 | 0.91 | 0.00 | 25 | 58.585 | 52.830 |
| C16:0 iso | 4.49 | 2.47 | 0.93 | 0.86 | 0.00 | 25 | 85.472 | 80.750 |
| C16:1 | 80.23 | 40.82 | 0.92 | 0.84 | 0.00 | 25 | 5.126 | 53.252 |
| C17:0 iso | 8.25 | 4.15 | 0.95 | 0.91 | 0.00 | 25 | 52.290 | 33.015 |
| C17:0 | 16.33 | 8.33 | 0.97 | 0.95 | 0.00 | 25 | 26.586 | 30.310 |
| C17:1 | 15.03 | 7.36 | 0.96 | 0.92 | 0.00 | 25 | 29.615 | 19.408 |
| C18:0 iso | 3.70 | 1.71 | 0.99 | 0.98 | 0.00 | 25 | 132.169 | −24.326 |
| C18:0 | 244.80 | 113.38 | 0.95 | 0.90 | 0.00 | 25 | 1.906 | −2.145 |
| C18:2 | 17.01 | 9.08 | 0.81 | 0.65 | 0.00 | 25 | 20.195 | 120.991 |
| C18:2 cj | 6.57 | 4.09 | 0.74 | 0.54 | 0.00 | 25 | 41.014 | 195.174 |
| C18:3 | 1.85 | 0.85 | 0.99 | 0.98 | 0.00 | 25 | 264.339 | −24.326 |
| C17:0 anteiso | 12.14 | 6.17 | 0.98 | 0.95 | 0.00 | 25 | 36.005 | 27.577 |
| C15:0 anteiso | 3.44 | 1.81 | 0.92 | 0.85 | 0.00 | 25 | 115.471 | 67.211 |
| C14:1 | 11.61 | 7.07 | 0.83 | 0.69 | 0.00 | 25 | 26.817 | 153.092 |
| C12:0 | 1.78 | 0.80 | 0.99 | 0.98 | 0.00 | 24 | 261.363 | −20.129 |

In all tables herein, the abbreviations used have the following meanings:

SD: standard deviation

r: correlation coefficient

p: statistical significance level

n: number of individuals tested

SFA: saturated fatty acids

MUFA: monounsaturated fatty acids

PUFA: polyunsaturated fatty acids

CLA: conjugated linoleic acids

ALA: alpha-linolenic acid

LA: linoleic acid

1. A method for determining the quantity of methane produced by a meat ruminant of a slaughtered animal comprising:
measuring the quantity of at least one fatty acid (FA) contained in a reference tissue sampled from said ruminant after said animal has been slaughtered;
calculating said quantity of methane according to an equation that is a function of said quantity of said FA and a category, age and weight of said animal, wherein the latter three criteria are determined at the time of the slaughter of said animal wherein said quantity is calculated according to the equation:
CH₄=((((age in months)*Coeff 1)+Coeff 2)*1000*lipid content*FA content))*1000)/weight of the animal,
in which:
the coefficients Coeff 1 and Coeff 2 are numbers whose value is a function of the nature of the reference tissue and the category of the animal, lipid content is a percentage of the weight of the reference tissue, FA content is a percentage of the total lipid content, weight is in kg and CH₄ is in g.

2. (canceled)

3. The determining method of claim 2, characterized in that said reference tissue is the longissimus dorsi muscle.

4. The determining method of either claim 2 or claim 3, characterized in that said determination of FA quantity is carried out by direct analysis of the reference tissue.

5. The determining method of claim 3, characterized in that said determination of FA quantity is carried out by analysis of another tissue, and then deduction of the FA quantity of said reference muscle by a prediction equation.

6. The method according to any one of the preceding claims, characterized in that said FA is palmitic acid C16:0.

7. The method according to claims 5 and 6 in combination, characterized in that said other tissue is selected from flank, skirt and silverside and that the quantity of palmitic acid in the longissimus dorsi (C16:0_{LD}) is given by one of the following equations:
C16:0_{LD}=0.884*C16:0_{Flank}+2.240(r²=0.855,n=48,p<0.001);
C16:0_{LD}=1.053*C16:0_{Skirt}+1.076(r²=0.78,n=67,p<0.001);
C16:0_{LD}=0.948*C16:0_{Silverside}+2.095(r²=0.70,n=25,p<0.001);
equations wherein C16:0_{Flank}, C16:0_{Skirt} and C16:0_{Silverside} are the palmitic acid contents of the corresponding tissues, r is the correlation coefficient, n is the number of samples tested and p is the significance level.

8. The method of any one of claims 1 to 5, characterized in that said FA is different from palmitic acid, but strongly correlated with palmitic acid, with a significance level (p) less than 0.01.

9. The method of claims 3 and 4 in combination, characterized in that said Coefficients 1 and 2 have the following values:

| | Coeff 1 | Coeff 2 |
|---|---|---|
| Young bovine | 1.511 ± 0.506 | −13.782 ± 5.0615 |
| Heifer | 0.555 ± 0.1905 | −4.807 ± 1.907 |
| Steer | 0.885 ± 0.278 | −7.522 ± 2.779 |
| Nursing cow | 0.582 ± 0.235 | 0 |

10. The method of claim 4, when claim 4 is dependent on claim 2 and the reference tissue is skirt, characterized in that said coefficients have the following values:

| | Coeff 1 | Coeff 2 |
|---|---|---|
| Young bovine | 0.5142 | −4.700 |
| Heifer | 0.3356 | −2.9042 |
| Steer | 0.3011 | −2.5596 |
| Nursing cow | 0.2671 | 0.000 |
